SEQUENCE LISTING (5 sequences)

SEQ ID NO: 1 - protein, 193 aa, H. sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 2 - protein, 18 aa, H. sapien
IVFSKDQFPEVYVPTVFE

SEQ ID NO: 3 - protein, 18 aa, H. sapien
PDTDVILMCFSIDSPDSL

SEQ ID NO: 4 - protein, 28 aa, H. sapien
LENIPEKWTPEVKHFCPNVPIILVGNKK

SEQ ID NO: 5 - protein, 16 aa, H. sapien
RQIKIWFQNRRMKWKK

(19) United States
(12) Patent Application Publication
(10) Pub. No.: US 2005/0209147 A1
(43) Pub. Date: Sep. 22, 2005

(54) Peptide inhibitors of RhoA signaling

(75) Inventors: Carlo Laudanna, Verona (IT); Eugene C. Butcher, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/035,714

(22) Filed: Jan. 14, 2005

Related U.S. Application Data
(60) Provisional application No. 60/537,142, filed on Jan. 16, 2004.

(51) Int. Cl. A61K 38/17; C07K 14/705

(52) U.S. Cl. 514/12; 530/350

(57) ABSTRACT

RhoA mediated signaling pathways are manipulated with specific domains of the RhoA protein that are translocation modified by conjugation or fusion to a transport domain. The compounds find use in therapeutic and research methods where it is desirable to selectively inhibit RhoA.

### BACKGROUND OF THE INVENTION

The concerted action of adhesion molecules and chemokine receptors regulates leukocyte extravasation from the blood and determines the specificity of the immune response. Chemokines are a superfamily of small, secreted, cytokines that are involved in a variety of immune and inflammatory responses, acting primarily as chemoattractants and activators of specific types of leukocytes. Chemokines mediate their activities by binding to target cell surface chemokine receptors, belonging to the large family of G protein-coupled, seven transmembrane (7 TM) domain receptors. Leukocytes have generally been found to express more than one receptor type, and the various receptors are known to exhibit overlapping ligand specificities. Besides directing chemotaxis, chemokines activate an extremely rapid and complex mode of integrin activation, consisting of heterodimer high affinity state and lateral mobility triggering.

Integrins, such as the β2 integrin lymphocyte function-associated antigen-1 (LFA-1), are subject to activation by inside-out signalling and, in turn, generate outside-in signalling leading to cell activation. These two signalling events may play a pivotal role in adhesion regulation. LFA-1 on resting T cells binds with low affinity its ligands ICAM-1, -2, -3. LFA-1 binding to ICAM-1 is increased by a large number of signalling pathways including TCR and chemokine receptors. This inside-out signal increases the interaction of LFA-1 with ICAM-1 on the outer side and with talin (and likely other actin-binding proteins) in the inner side.

Outside-in signals from integrins can modulate the activity/affinity of other integrins and contribute to T cell activation. Synergy between LFA-1 and TCR has also been described in the induction of inositol-1,4,5-triphosphate (IP3) generation and Ca²⁺ flux. In pre-activated CD4 cells, LFA-1 engagement alone can induce Ca²⁺ influx and PLCγ₁ phosphorylation.

These modalities of integrin activation play a cooperative role in mediating LFA-1-dependent lymphocyte immediate arrest on ICAM-1 under physiologic flow conditions. In particular, induction of rapid LFA-1 lateral mobility on the plasma membrane has been shown to mediate lymphocyte arrest to surfaces presenting a low density of ICAM-1. This suggests that LFA-1 lateral mobility allows the adaptation of lymphocytes to blood vessels presenting a variable expression level of integrin ligand.

Signaling pathways controlling LFA-1 activation are largely unknown. Recent data show the involvement of phosphatidylinositol 3(—OH) kinase (PI(3)K), Cytohesin-1 and Rap1 in LFA-1 lateral mobility induced by chemokines in lymphocytes. However, signaling events controlling the rapid induction of LFA-1 high affinity state by chemokines are completely unidentified.

RhoA is a member of the Ras homology family of small GTPases. These proteins cycle from their active (GTP-bound) to their inactive (GDP-bound) conformation by hydrolyzing GTP to GDP. RhoA's functions in the cell are primarily related to cytoskeletal regulation. Recent studies have shown its indirect involvement in myosin phosphorylation and cellular responses to stress, such as the formation of focal adhesions and actin stress fibers. It has also been shown to be directly related to myosin chain elongation, actin filament rearrangement, gene expression, cell-shape determination and cell proliferation.

RhoA has three known main effectors, which include the ROCK I, II family, which are kinases that cause actomyosin contraction, transformation, and transcription of the SRF gene. Also, these effectors show scaffolding properties that function to polymerize actin and affect the formation of microtubules. The second effector is the PRK1/PKN proteins that cause endocytosis. And lastly RhoA binds to the effector Citron causing cytokinesis.

Previous data have implicated the small GTPase RhoA and the atypical ζ PKC in chemoattractant-induced β1 and β2-integrin mediated leukocyte adhesion. The discovery that chemokines activate in lymphocytes a complex modality of integrin activation raises the hypothesis that RhoA and ζ PKC may control specific modalities of LFA-1 triggering. In addition, RhoA activates several downstream effectors and recently it was shown that the ability of RhoA to diversify signaling pathways depends on engagement of distinct effector domains.

Methods of manipulating RhoA signaling are of interest for clinical and research methods, and are of particular interest for manipulation of immune responsiveness.

### SUMMARY OF THE INVENTION

Compositions and methods are provided for specific manipulation of RhoA mediated signaling pathways. Specific effector domains of the RhoA protein are modified by conjugation or fusion to a plasma membrane translocating domain. The resulting translocation modified peptides, for example, alter leukocyte adhesion and migration through RhoA-dependent control of LFA-1 high affinity state and lateral mobility induction. The compounds find use in therapeutic and research methods where it is desirable to selectively inhibit RhoA.

The compounds of the present invention find use as anti-inflammatory agents in the inhibition of leukocyte adhesion and migration. Anti-inflammatory activity may be locally delivered, e.g. for the treatment of autoimmune diseases such as psoriasis; rheumatoid arthritis, etc.; or may be systemically delivered, e.g. in the treatment of multiple sclerosis, to block immune reaction during transplantation, etc. The activity of the peptides in blocking lymphocyte recruitment in brain microvessels indicates a therapeutic use in the treatment of multiple sclerosis.

The compounds also find use in the inhibition of RhoA mediated pathways involved in cancer proliferation and/or metastasis; to prevent fibrosis and wound retraction after surgical intervention (peptides block ROCK activation by RhoA); to prevent, or reduce, formation of lymphocyte syncytia during HIV infection; and the like.

### BRIEF DESCRIPTION OF THE DRAWINGS

FIG. 1A-D. P1-RhoA fusion domains are effective inhibitors of RhoA-dependent signaling. FIG. 1(A) RhoA binding to Citron-, Rhotekin- and ROCK-RBD in presence of buffer (Control) or 20 μg of Penetratin-1 (P1) or 23-40, 75-92 and 92-119 P1-RhoA fusion domains. A protein immunoblot of anti-RhoA is shown. One representative experiment of two. FIG. 1(B) Densitometric analysis of the immunoblot showed in (A). FIG. 1(C) Swiss 3T3 mouse fibroblasts were treated for 4 hours at 37° C. with 500M of Penetratin-1 (P1) or different P1-RhoA fusion domains and then stimulated for 10 min with 25 ng/ml lysophosphatidic acid (LPA). Shown are confocal microscopy images. FIG. 1(D) Human polymorphonuclear neutrophils were treated for 2 hours at 37° C. with 50 μM of P1 or different P1-RhoA fusion domains and then stimulated with 10 ng/ml PMA or 100 nM formyl-Met-Leu-Phe (fMLP). Stimulation was performed under stirring at 37° C. Shown are nmoles of released H₂O₂. Values are means from three experiments. Error bars are SDs.

FIG. 2A-H. RhoA control LFA-1 high affinity state and lateral mobility triggering by CCL21. FIG. 2(A) ICAM-1 was immobilized at the indicated site densities. Lymphocytes were treated at 37° C. for 60 min with buffer (n.a. and C) or with the indicated μM doses of P1 or different P1-RhoA fusion domains and then stimulated for 2 min with buffer (n.a.=no agonist) or with 1 μM CCL21. Values are mean counts of adherent cells in 6 to 9 experiments. Error bars are SDs. *P<0.01. FIG. 2(B) Lymphocytes were stimulated at 37° C. under stirring with 1 μM CCL21 for the indicated time; time 0 corresponds to no agonist. A protein immunoblot of anti-RhoA in lysates (up) and precipitates (down) is shown. One representative experiment of three. FIG. 2(C) Lymphocytes were treated at 37° C. for 60 min with 100 μM of ¹²⁵I P1 or P1-RhoA fusion domains. Values are mean numbers of internalized molecules per cell in 3 experiments. Error bars are SDs. FIG. 2(D) RhoA controls the induction of LFA-1 high affinity state by CCL21. Lymphocytes were treated at 37° C. for 60 min with buffer (n.a. and control), with 100 μM of P1 or different P1-RhoA fusion domains or FIG. 2(E) with the indicated concentrations of P1-23/40 RhoA domain and then stimulated for 2 min with buffer (n.a.=no agonist) or with 1 μM CCL21. The mean CPM from ¹²⁵I-ICAM-1 in three experiments is shown. Error bars are SDs. *P<0.01. FIG. 2(F) RhoA controls the induction of LFA-1 rapid lateral mobility on the plasma membrane induced by CCL21. Lymphocytes were treated as described for FIG. 2D, and then stimulated at 37° C. for 2 min. with 1 μM CCL21. Confocal images of LFA-1 surface distribution are shown. Arrows indicate LFA-1 clusters. FIG. 2(G) ICAM-1 was immobilized at the indicated site densities. Lymphocytes were treated at 37° C. 30 min with buffer (no agonist and control) or with 50 μM Y27632 and then stimulated for 2 min. with buffer (no agonist) or with 1 μM CCL21. Values are the mean counts of adherent cells in 3 experiments. Error bars are SDs. FIG. 2(H) Lymphocytes were treated with 50 μM Y27632. LFA-1 affinity triggering was evaluated as described for FIG. 2D. Values from 3 experiments. Error bars are SDs.

FIG. 3A-D. ζ PKC is involved in LFA-1 activation by CCL21. FIG. 3(A) ICAM-1 was immobilized at the indicated site densities. Lymphocytes were treated at 37° C. for 60 min with buffer (n.a. and control) or with 50 μM of a scramble peptide (scr) or with the indicated doses of ζ PKC myristoylated pseudosubstrate peptides and then stimulated for 3 min with buffer (n.a.=no agonist) or with 1 μM CCL21. Values are mean counts of adherent cells in 5 experiments. Error bars are SDs. FIG. 3(B) ICAM-1 was immobilized at the indicated site densities. Lymphocytes were treated at 37° C. for 60 min with buffer (n.a. and control) or with the indicated dose of various PKC myristoylated pseudosubstrate peptides and then stimulated for 3 min with buffer (n.a.=no agonist) or with 1 μM CCL21. Values are mean counts of adherent cells in 4 experiments. Error bars are SDs. FIG. 3(C) Lymphocytes were treated at 37° C. for 30 min. with buffer (no agonist and control) or with 150 nM Wortmannin or 30 μM LY294002 and then stimulated at 37° C. under stirring for 30 or 60 seconds with buffer (no agonist) or with 1 μM CCL21. No agonist w/o MBP is radioactivity in absence of exogenous substrate and is measurement of ζ PKC auto-phosphorylating activity in non-stimulated lymphocytes. Values are the mean counts of two experiments performed in duplicate. Error bars are SDs. FIG. 3(D) Lymphocytes were treated at 37° C. for 30 min. with buffer (no agonist and control), with 100 μM of P1 or P1-RhoA fusion domains or with 150 nM Wortmannin and then stimulated at 37° C. under stirring for 30 seconds with buffer (no agonist) or with 1 μM CCL21. Shown are protein immunoblots of cytosolic (C), light membrane (M) and particulate (P) fractions separated on sucrose gradient and probed with anti-ζ PKC Ab.

FIG. 4A-B. ζ PKC control LFA-1 lateral mobility but not high affinity state triggering by CCL21. FIG. 4(A) Lymphocytes were treated at 37° C. for 60 min with buffer (no agonist and control), or with 50 μM scramble peptide (scr) or ζ PKC myristoylated pseudosubstrate peptide and then stimulated for 2 min with buffer (no agonist) or with 1 μM CCL21. The mean CPM from ¹²⁵I-ICAM-1 is shown Values are counts from a representative experiment of three. FIG. 4(B) Lymphocytes were treated as in (A) and then stimulated at 37° C. for 2 min. with 1 μM CCL21. Confocal images of LFA-1 surface distribution are shown. Arrows indicate LFA-1 clusters.

FIG. 5. LFA-1 high affinity state mediates lymphocyte homing to HEV in Peyer's patches. Intravital microscopy was performed in Peyer's patche high endothelial venules. Lymphocytes were treated with buffer (control) or with 50 μM P1 and P1-RhoA fusion domains, with 50 μM Y27632 or with 100 μM of ζ PKC myristoylated pseudosubstrate peptide. Values are the mean percentage of total interacting cells in three experiments. Error bars are SDs. *P<0.01

FIG. 6A-C. The distinct roles of RhoA and ζ PKC in the different modalities of rapid LFA-1 activation by chemokines. FIG. 6(A) Domain organization of RhoA and ζ PKC showing the effector regions of RhoA (aa. 23-40 (A), 75-92 and 92-119 (B)), and the inhibitory pseudosubstrate domain of ζ PKC (aa. 113-129). FIG. 6(B) The plasma membrane translocating peptides displaying inhibitory capability on LFA-1 activation. The 23-40 (1) and 92-119 (2) RhoA effector regions were fused to Penetratin-1. A myristic acid was added to N-terminal of the pseudosubstrate region of ζ PKC (3). (C) Induction of LFA-1 high affinity state by chemokines is controlled by the signaling activity of 23-40 downstream effector region of RhoA (marked with A). Induction of rapid LFA-1 lateral mobility is controlled by ζ PKC and by further signals generated by the 92-119 downstream effector region of RhoA (marked with B). The capability of ζ PKC to control rapid LFA-1 lateral mobility depends on translocation to the plasma membrane, which is controlled by RhoA 23-40 downstream effector region, as well as by ζ PKC kinase activity. ζ PKC appears to be a PI(3)K as well as RhoA downstream effector mediating RhoA-dependent LFA-1 lateral mobility induced by chemokines. (1) inhibition by the P1-RhoA 2340 fusion domain; (2) inhibition by the P1-RhoA 92-119 fusion domain; (3) inhibition by the myristoylated peptide with sequence identical to ζ PKC pseudosubstrate region.

FIG. 7. Effect of Rho-A derived Trojan peptide on in vivo lymphocyte recruitment in inflamed brain microvessels.

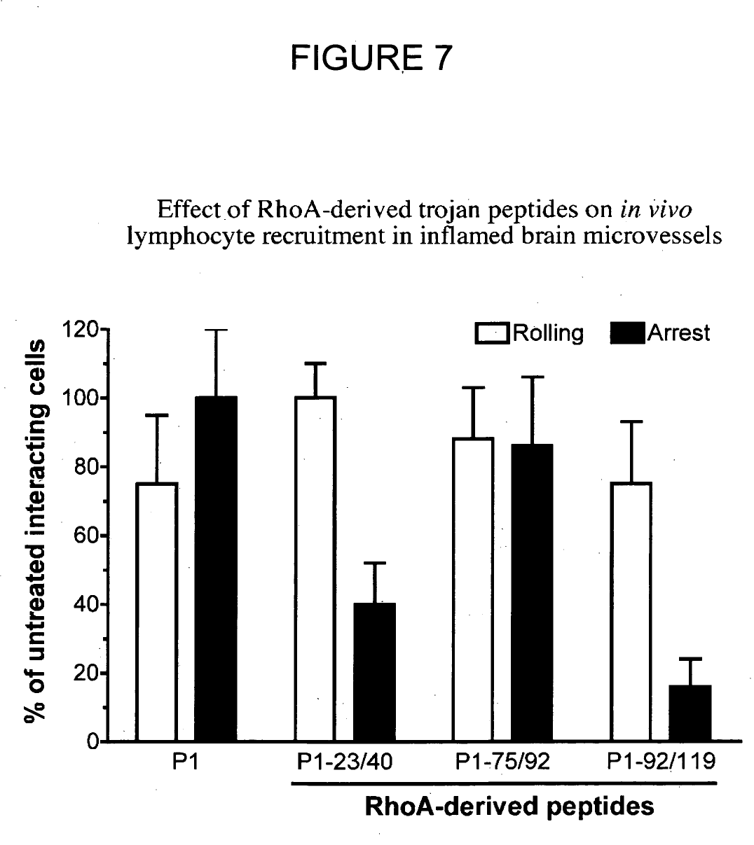

### DETAILED DESCRIPTION OF THE EMBODIMENTS

RhoA mediated signaling pathways are inhibited by the administration of peptides comprising specific domains of the RhoA protein. The RhoA derived peptides may be translocation modified, e.g. by conjugation or fusion to a transport domain, to provide for transport across a cell membrane.

Inhibition of RhoA signaling finds particular use in the modulation of leukocyte adhesion and migration, through RhoA control of LFA-1 high affinity state and lateral mobility induction. For example, the subject peptides are administered systemically or locally to inhibit inflammation and other immunological activity. The peptides have research uses in the specific investigation of RhoA signaling pathways, providing the benefit that it is not necessary to genetically modify the targeted cells, i.e. one can treat a cell with exogenous peptides. The peptides are superior to traditional pharmacological inhibition of RhoA, e.g. by Clostridium botulinum C3 convertase, in that the peptides are much easier to use and act as domain-selective inhibitors.

The compounds also find use in the inhibition of RhoA mediated pathways involved in cancer proliferation and/or metastasis; to prevent fibrosis and wound retraction after surgical intervention (peptides block ROCK activation by RhoA); to prevent, or reduced, formation of lymphocyte syncytia during HIV infection; and the like.

Chemokines regulate rapid leukocyte adhesion by triggering a complex modality of integrin activation. It is shown herein that specific domains of the small GTPase RhoA, and the atypical ζ PKC, differently control lymphocyte LFA-1 high affinity state and rapid lateral mobility induced by chemokines. Activation of LFA-1 high affinity state and lateral mobility is controlled by RhoA through the activity of distinct effector domains, demonstrating that RhoA is a central point of diversification of signaling pathways leading to both modalities of LFA-1 triggering. Blockade of the 23-40 RhoA domain prevents induction of LFA-1 high affinity state as well as lymphocyte arrest in Peyer's patch high endothelial venules. Thus, RhoA controls the induction of LFA-1 high affinity state by chemokines independently of ζ PKC and this is critical to support chemokine-regulated homing of circulating lymphocytes.

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

### Peptides

Peptides of the present invention comprise RhoA effector domains, and may further comprise (1) a transport domain (T), which is covalently or non-covalently linked to said RhoA domain (R); having the structure TXR, or RXT, where X may be a linker, a peptide bond, or one or more amino acids, e.g. a peptide linker of G, A, etc.

The RhoA peptides may be referred to by the amino acid residues, which are based on the human sequence, e.g. as set forth in Genbank accession number NP_{—}001655 and as described by Yeramian et al., (1987) Nucleic Acids Res. 15 (4):1869. The sequence of the complete RhoA polypeptide is provided for convenience in the Seqlist, as SEQ ID NO:1. The RhoA peptides may also be referred to with respect to the sequences provided herein, and variants and derivatives thereof. It will be understood by one of skill in the art that conservative substitutions and other modifications may be made to the sequence, and that RhoA peptides from other species may also find use, where the appropriate alignment is made with the human sequence.

The RhoA effector domains include the following exemplary amino acid sequences: Domain I, SEQ ID NO:1, res. 23-40; comprises the amino acid sequence IVFSKDQFPEVYVPTVFE (SEQ ID NO:2). Domain II, SEQ ID NO:1, res. 75-92; comprises the amino acid sequence PDTDVILMCFSIDSPDSL (SEQ ID NO:3). Domain III, SEQ ID NO:1, res. 92-119; comprises the amino acid sequence LENIPEKWTPEVKHFCPNVPIILVGNKK (SEQ ID NO:4).

The domains have distinct activities. RhoA controls LFA-1 conformational change and lateral mobility by chemokines through the activity of Domain I, which is critical to the induction of the LFA-1 high affinity state and heterodimer lateral mobility regulation, and Domain II, which is involved in LFA-1 lateral mobility induction. Lymphocytes rely on activation of LFA-1 high affinity state to home to secondary lymphoid organs. The Domain I peptides described herein effectively block LFA-1 high affinity triggering, and prevents LFA-1-dependent arrest of naïve lymphocytes on high endothelial venules. Alternatively, selective blockade of the 92-119 RhoA domain exclusively inhibits LFA-1 lateral mobility, without affecting lymphocyte homing to secondary lymphoid organs. Domain I also blocks ζ PKC translocation to the plasma membrane, suggesting a direct interaction between RhoA and ζ PKC, and establishing ζ PKC as a novel direct downstream effector of RhoA.

Specifically, Domain I, but not II or III, blocks the binding of RhoA to Citron; binding to Rhotekin was blocked by Domain II, but not I or III; and binding to ROCK is inhibited by all three domains. As ROCK controls, the accumulation of actin stress fibers induced by lysophosphatidic acid (LPA) in fibroblasts, the biological activity of the three RhoA fusion domains is supported by their capability to prevent LPA-induced stress fibers accumulation.

For use in the subject methods, the RhoA domain is a peptide of at least about 12 amino acids in length, more usually at least about 16 amino acids in length, and not more than about 30 amino acids in length. The carboxy terminus of the peptide may be in the form of a free acid, or an amide, preferably an amide.

The sequence of the RhoA domain polypeptide may be altered from those provided herein in various ways known in the art to generate targeted changes in sequence. The altered peptide will usually be substantially similar to the sequences provided herein, i.e. will differ by one amino acid, and may differ by two or more amino acids. The sequence changes may be substitutions, insertions or deletions.

The RhoA domain peptide may be joined to a wide variety of other oligopeptides or proteins for a variety of purposes. For instance, the transport domain (T) can be antennapedia-derived penetratin-1 and its derivative modifications, the HIV-derived TAT and its derivative or any other amino acid sequence able to allow translocation though the plasma membrane. Moreover, various post-translational modifications may be achieved. For example, by employing the appropriate coding sequences, one may provide myristoylation, farnesylation or prenylation. In this situation, the peptide will be bound to a lipid group at a terminus, so as to be able to be bound to a lipid membrane, such as a liposome.

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids.

The subject peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like. If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In one embodiment of the invention, the peptide consists essentially of a polypeptide sequence set forth herein. By “consisting essentially of” in the context of a polypeptide described herein, it is meant that the polypeptide is composed of the sequence set forth in the seqlist, which sequence may be flanked by one or more amino acid or other residues that do not materially affect the basic characteristic(s) of the polypeptide.

Transport Domain. A number of transport domains are known in the art and may be used in the present invention, including peptides, peptidomimetics, and non-peptide carriers. In one embodiment, the transport peptide is derived from the third alpha helix of Drosophila melanogaster transcription factor Antennapaedia, referred to as penetratin, which comprises the amino acid sequence RQIKIWFQNRRMKWKK (SEQ ID NO:5). In another embodiment, the transport peptide comprises the HIV-1 tat basic region amino acid sequence, which may include, for example, amino acids 49-57 of naturally-occurring tat protein. Other transport domains include poly-arginine motifs, for example, the region of amino acids 34-56 of HIV-1 rev protein. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids).

Linkers. The attachment of the RhoA domain to the transport domain may utilize any means that produces a link between the constituents that is sufficiently stable to withstand the conditions used, and that does not alter the function of either constituent. High affinity non-covalent, e.g. biotin and avidin or streptavidin; or covalent bonds are preferred. In one embodiment of the invention, the linker is a cleavable linker containing a bond that is cleaved by an enzyme, energy source or solvent, e.g. esters, amides, disulfides, etc.

For example, recombinant techniques can be used to covalently attach the transport domain to the RhoA domain (cargo), e.g. by genetically creating a fusion, and introducing the genetic construct encoding the fusion protein into a cell capable of expressing it. Alternatively, such a fusion protein can be synthesized chemically as a single amino acid sequence.

Chemical groups that find use in linkage include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hyrodrazone, lipids, and the like, as known in the art. Ester and disulfide linkages are preferred if the linkage is to be readily degraded in the cytosol after transport of the substance. Various functional groups (hydroxyl, amino, halogen, etc.) can be used to attach the cargo to the transport domain.

The linkage may also comprise spacers, e.g. amino acid spacers, alkyl spacers, which may be linear or branched, usually linear, and may include one or more unsaturated bonds; usually having from one to about 300 carbon atoms; more usually from about one to 25 carbon atoms; and may be from about three to 12 carbon atoms.

There are situations where it is desirable to cleave the linker between the transport domain and cargo. In one embodiment of the invention, the cargo is linked through a cleavable linker to the transport domain, such that on entry into the cell the cargo is released. The cleavable moiety may be provided in the linkage group, or in the spacer between the linking groups. The cleavable moiety is cleaved by an agent, which may be biological, e.g. enzymatic, chemical or physical, e.g. temperature, ionicity, light, pH, etc. One or more specific recognition sites may be present.

Enzymatically cleavable linkages of interest include nucleic acids, e.g. DNA/DNA oligonucleotide hybrids; DNA-RNA oligonucleotide hybrids; RNA-RNA oligonucleotide hybrids; which are cleavable with nucleases; oligosaccharides, which are cleavable with glycosidases; polypeptides, which are cleavable by proteases; lipids; etc. Each of these may be cleaved at specific, or non-specific sites.

Polypeptides of interest as cleavable linkers include those having a recognition site for a protease present in the targeted cell. Cleavable oligosaccharide linkers may include dextran having an α(1 [symbol] 6) glycosidic linkage; cellulose having a β(1 [symbol] 4) glycosidic linkage; amylose; pectin; chitin, etc. The linker may also be cleavable through light, i.e. photocleavable, or the linker may be chemically cleavable, e.g. acid or base labile. In such linkers, the linker will comprise a cleavable moiety that is either photo or chemically cleavable. Photocleavable or photolabile moieties that may be incorporated into the linker include: o-nitroarylmethine and arylaroylmethine, as well as derivatives thereof, and the like. Photocleavable linkages that can be activated by exposure to light are described, for example, in U.S. Pat. No. 5,739,386.

Formulations. The compounds of this invention can be incorporated into a variety of formulations for therapeutic administration. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, aerosols and nanoparticles. As such, administration of the compounds can be achieved in various ways, including oral, buccal, rectal, parenteral, intraperitoneal, intradermal, transdermal, intracheal, etc., administration. The conjugate of transport domain and RhoA may be systemic after administration or may be localized by the use of an implant that acts to retain the active dose at the site of implantation.

The compounds of the present invention can be administered alone, in combination with each other, or they can be used in combination with other known compounds, e.g. in a cocktail of RhoA domains. In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the targeted site, so that the local concentration of active agent is increased relative to the rest of the body.

The term “unit dosage form,” as used herein, refers to physically discrete units suitable as unitary dosages for human and animal subjects, e.g. mammals including equine, canine, feline, bovine, caprine, murine, primate, etc., each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

In the certain methods of the invention, the subject peptide compounds are administered systemically or locally to alter the trafficking behavior of leukocytes. Trafficking, or homing, is used herein to refer to the biological activities and pathways that control the localization of leukocytes in a mammalian host. Such trafficking may be associated with disease, e.g. inflammation, allergic reactions, etc., or may be part of normal biological homeostasis.

Local administration that provides for a prolonged localized concentration, which may utilize sustained release implants or other topical formulation, is of particular interest. In one embodiment of the invention, the translocation-modified rhoA peptide acts to decrease the local concentration of responsive leukocytes. In vivo uses of the method are of interest for therapeutic and investigational purposes. In vitro uses are of interest for determination of physiological pathways, and the like.

LFA-1 is primarily expressed by lymphocytes, including memory T cells, granulocytes, monocytes and macrophages. The mononuclear phagocyte system is comprised of both circulating and resident populations of cells. The circulating component is the monocyte. Upon migration into tissues these are referred to as histiocytes or tissue macrophages. The major resident macrophages include: Sinusoidal lining cells of the spleen, lymph nodes, liver, and bone marrow; connective tissue histiocytes; mobile macrophages on serosal surfaces; alveolar macrophages within the lung; microglia in the nervous system; and mesangial macrophages within renal glomeruli. Macrophages produce a variety of substances that are involved in inflammation. Mast cells are important mediators of certain allergic reactions. Mast cell membranes have abundant IgE receptor sites, anywhere from 30,000 to 500,000 per cell. If a particular antigen incites an IgE response, the resulting IgE is bound to the IgE receptors on mast cell surfaces via the Fc portion of the immunoglobulin molecule. Interaction of an antigen with surface-bound IgE results in cross-linking of the IgE molecules, mast cell activation, and ultimately mast cell degranulation.

Lymphocytes are another class on mononuclear cell of interest for the methods of the invention. Lymphocytes may be broadly divided into B cells, T cells and natural killer cells. T cells and B cells are able to give rise to memory cells, as well as effector cells. Circulating T cells are small, round-shaped cells with very little cytoplasm and a number of protrusions (microvilli) on the plasma membrane. During extravasation, T cells undergo major morphological changes as they adhere, spread and eventually transmigrate through the endothelium. The recirculation pattern of T cells is highly regulated by the modulated expression and function of specific receptor-ligand pairs on the cell surface of T and endothelial cells, respectively.

Naive T cells are primed by specialized antigen presenting cells in secondary lymphoid tissues. Upon antigen recognition they undergo clonal amplification and progressively acquire differentiated functions. Cytolytic T cells are CD8+, and can secrete a number of lytic proteins. CD4⁺ T cells mature into two major subsets of effectors, based on the cytokines they produce. Th1 and Th2 cells enhance cellular and humoral adaptive responses to antigen. A third subset comprises T regulatory cells (Tr), which negatively control the above responses due to the production of selected cytokines.

Maturation of T cells includes the acquisition of a memory phenotype by a subpopulation of clonally expanded T cells that progressively exit the cell cycle and revert to a quiescent state. Memory may be long-lasting, and is both antigenic and topographic, the latter being provided by the expression of defined arrays of chemotactic and homing receptors. These dictate the recirculation pattern of memory versus naive T cells. To ensure maximal efficiency and sensitivity in antigen recognition and elimination, naive cells preferentially recirculate through secondary lymphoid organs, while memory and effector cells patrol peripheral tissues and re-enter the blood via the afferent lymphatics.

The translocation-modified rhoA peptide may be delivered as a bolus, or may provide for a localized concentration by use of a sustained release formulation. Preliminary doses can be determined according to animal tests, and the scaling of dosages for human administration can be performed according to art-accepted practices. A variety of sustained release formulations are known and used in the art. For example, biodegradable or bioerodible implants may be used. The implants may be particles, sheets, patches, plaques, fibers, microcapsules, nanoparticles and the like and may be of any size or shape compatible with the selected site of insertion. Characteristics of the polymers will include biodegradability at the site of implantation, compatibility with the agent of interest, ease of encapsulation, the half-life in the physiological environment, water solubility, and the like.

Another approach involves the use of an implantable drug delivery device. Examples of such implantable drug delivery devices include implantable diffusion systems (see, e.g., subdermal implants (such as NORPLANT™) and other such systems, see, e.g., U.S. Pat. Nos. 5,756,115; 5,429,634; 5,843,069). These implants generally operate by simple diffusion, e.g., the active agent diffuses through a polymeric material at a rate that is controlled by the characteristics of the active agent formulation and the polymeric material. Alternatively, the implant may be based upon an osmotically-driven device to accomplish controlled drug delivery (see, e.g., U.S. Pat. Nos. 3,987,790, 4,865,845; 5,057,318; 5,059,423; 5,112,614; 5,137,727; 5,234,692; 5,234,693; and 5,728,396). These osmotic pumps generally operate by imbibing fluid from the outside environment and releasing corresponding amounts of the therapeutic agent.

Immune responses, particularly responses associated with LFA-1 mediated trafficking, are inhibited by administration of the subject peptides. The treatment of ongoing disease, to stabilize or improves the clinical symptoms of the patient, is of particular interest. Immune related diseases include autoimmune diseases, in which the immune response aberrantly attacks self-antigens, examples of which include but are not limited to multiple sclerosis (MS), rheumatoid arthritis (RA), type I autoimmune diabetes (IDDM), and systemic lupus erythematosus (SLE); allergic diseases in which the immune system aberrantly attacks molecules such as pollen, dust mite antigens, bee venom, peanut oil and other foods, etc.; and tissue transplant rejection in which the immune system aberrantly attacks antigens expressed or contained within a grafted or transplanted tissue, such as blood, bone marrow cells, or solid organs including hearts, lungs, kidneys and livers.

Rheumatoid arthritis (RA) is a chronic autoimmune inflammatory synovitis affecting 0.8% of the world population. Current therapy for RA utilizes therapeutic agents that non-specifically suppress or modulate immune function. Such therapeutics, including the recently developed TNFα antagonists, are not fundamentally curative, and disease activity rapidly returns following discontinuation of therapy. Tremendous clinical need exists for fundamentally curative therapies that do not cause systemic immune suppression or modulation.

Degenerative joint diseases may be inflammatory, as with seronegative spondylarthropathies, e.g. ankylosing spondylitis and reactive arthritis; rheumatoid arthritis; gout; and systemic lupus erythematosus. The degenerative joint diseases have a common feature, in that the cartilage of the joint is eroded, eventually exposing the bone surface. Destruction of cartilage begins with the degradation of proteoglycan, mediated by enzymes such as stromelysin and collagenase, resulting in the loss of the ability to resist compressive stress. Alterations in the expression of adhesion molecules, such as CD44 (Swissprot P22511), ICAM-1 (Swissprot P05362), and extracellular matrix protein, such as fibronectin and tenascin, follow. Eventually fibrous collagens are attacked by metalloproteases, and when the collagenous microskeleton is lost, repair by regeneration is impossible.

There is significant immunological activity within the synovium during the course of inflammatory arthritis. While treatment during early stages is desirable, the adverse symptoms of the disease may be at least partially alleviated by treatment during later stages. Clinical indices for the severity of arthritis include pain, swelling, fatigue and morning stiffness, and may be quantitatively monitored by Pannus criteria. Disease progression in animal models may be followed by measurement of affected joint inflammation. Therapy for inflammatory arthritis may combine the subject treatment with conventional NSAID treatment. Generally, the subject treatment will not be combined with such disease modifying drugs as cyclosporin A, methotrexate, and the like.

A quantitative increase in myelin-autoreactive T cells with the capacity to secrete IFN-gamma is associated with the pathogenesis of MS and EAE, suggesting that autoimmune inducer/helper T lymphocytes in the peripheral blood of MS patients may initiate and/or regulate the demyelination process in patients with MS. The overt disease is associated with muscle weakness, loss of abdominal reflexes, visual defects and paresthesias. During the presymptomatic period there is infiltration of leukocytes into the cerebrospinal fluid, inflammation and demyelination. Family histories and the presence of the HLA haplotype DRB1*1501, DQA1*0102, DQB1*0602 are indicative of a susceptibility to the disease. Markers that may be monitored for disease progression are the presence of antibodies in the cerebrospinal fluid, “evoked potentials” seen by electroencephalography in the visual cortex and brainstem, and the presence of spinal cord defects by MRI or computerized tomography. Treatment during the early stages of the disease will slow down or arrest the further loss of neural function.

Human IDDM is a cell-mediated autoimmune disorder leading to destruction of insulin-secreting beta cells and overt hyperglycemia. T lymphocytes invade the islets of Langerhans, and specifically destroy insulin-producing β-cells. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic beta cell function. The disease progression may be monitored in individuals diagnosed by family history and genetic analysis as being susceptible. The most important genetic effect is seen with genes of the major histocompatibility locus (IDDM1), although other loci, including the insulin gene region (IDDM2) also show linkage to the disease (see Davies et al, supra and Kennedy et al. (1995) Nature Genetics 9:293□298).

Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration. After the onset of overt diabetes, patients with residual beta cell function, evidenced by the plasma persistence of insulin C-peptide, may also benefit from the subject treatment, to prevent further loss of function.

Allergy, or atopy is an increased tendency to IgE-based sensitivity resulting in production of specific IgE antibody to an immunogen, particularly to common environmental allergens such as insect venom, house dust mite, pollens, molds or animal danders. Allergic responses are antigen specific. The immune response to the antigen is further characterized by the over-production of Th2-type cytokines, e.g. IL-4, IL-5 and IL-10, by the responding T cells. The sensitization occurs in genetically predisposed people after exposure to low concentrations of allergen; cigarette smoke and viral infections may assist in the sensitization process.

Included in the group of patients suffering from atopy are those with asthma-associated allergies. About 40% of the population is atopic, and about half of this group develop clinical disease ranging from trivial rhinitis to life-threatening asthma. After sensitization, continuing exposure to allergens leads to a significant increase in the prevalence of asthma. Ninety percent of children and 80% of adults with asthma are atopic. Once sensitization has occurred, re-exposure to allergen is a risk factor for exacerbations of asthma. Effective management of allergic asthma includes pharmacological therapy and allergen avoidance. The specific physiological effects of asthma associated allergies include airway inflammation, eosinophilia and mucus production, and antigen-specific IgE and IL4 production.

Immune rejection of tissue transplants, including lung, heart, liver, kidney, pancreas, and other organs and tissues, is mediated by immune responses in the transplant recipient directed against the transplanted organ. Allogeneic transplanted organs contain proteins with variations in their amino acid sequences when compared to the amino acid sequences of the transplant recipient. Because the amino acid sequences of the transplanted organ differ from those of the transplant recipient they frequently elicit an immune response in the recipient against the transplanted organ. Rejection of transplanted organs is a major complication and limitation of tissue transplant, and can cause failure of the transplanted organ in the recipient. The chronic inflammation that results from rejection frequently leads to dysfunction in the transplanted organ. Transplant recipients are currently treated with a variety of immunosuppressive agents to prevent and suppress rejection. These agents include glucocorticoids, cyclosporin A, Cellcept, FK-506, and OKT3.

LFA-1 triggering is also involved in the formation of syncytia following infection with HIV-1. The subject peptides, particularly Domain I peptide, may be administered alone, or in combination with other antiviral therapies to diminish HIV-1 infection.

In addition to anti-inflammatory activity, the peptides of the invention find use in inhibiting other activities of RhoA. RhoA and RhoC (which has an identical sequence to the Domain I peptide) are highly over-expressed in certain cancers, which include breast cancer; bladder cancer, ovarian carcinoma and pancreatic cancer. The RhoA function in these cells can be blocked by administration of the subject peptides.

The peptides of the invention may also be utilized in vitro, for example in the determination of signaling pathways affected by RhoA signaling. For example, factors, compounds, cells, etc. may be added to a cell culture in the absence and presence of the subject peptides, in order to determine in involvement of RhoA; and the specific domains of RhoA that are involved. Cell types of interest include endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells; etc.; adipocytes, cells involved with particular organs, such as thymus, endocrine glands, pancreas, brain, such as neurons, glia, astrocytes, dendrocytes, etc. and genetically modified cells thereof. Hematopoietic cells will be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCI (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free.

There are established protocols for the culture of diverse cell types. Such methods are described in the following: Animal Cell Culture Techniques (Springer Lab Manual), Clynes (Editor), Springer Verlag, 1998; Animal Cell Culture Methods (Methods in Cell Biology, Vol 57, Barnes and Mather, Eds, Academic Press, 1998; Harrison and Rae, General Techniques of Cell Culture (Handbooks in Practical Animal Cell Biology), Cambridge University Press, 1997; Endothelial Cell Culture (Handbooks in Practical Animal Cell Biology), Bicknell (Editor), Cambridge University Press, 1996; Human Cell Culture, Cancer Cell Lines Part I: Human Cell Culture, Masters and Palsson, eds., Kluwer Academic Publishers, 1998; Human Cell Culture Volume II—Cancer Cell Lines Part 2 (Human Cell Culture Volume 2), Masters and Palsson, eds., Kluwer Academic Publishers, 1999; Wilson, Methods in Cell Biology: Animal Cell Culture Methods (Vol 57), Academic Press, 1998; Current Protocols in Immunology, Coligan et al., eds, John Wiley & Sons, New York, N.Y., 2000; Current Protocols in Cell Biology, Bonifacino et al., eds, John Wiley & Sons, New York, N.Y., 2000.

The cell surface expression of various surface and intracellular markers, including protein, lipid, nucleic acid, e.g. genetic markers, and carbohydrate is known for a large number of different types of cells, and can be used as a reference for establishing the exact phenotype of cells; for determining the manner in which cells respond to an agent.

Those of skill will readily appreciate that dose levels can vary as a function of the specific compound, the severity of the symptoms and the susceptibility of the subject to side effects. Some of the specific complexes are more potent than others. Preferred dosages for a given agent are readily determinable by those of skill in the art by a variety of means. A preferred means is to measure the physiological potency of a given compound.

### EXPERIMENTAL

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which scope will be determined by the language in the claims.

It must be noted that as used herein and in the appended claims, the singular forms “a”, “and”, and “the” include plural referents unless the context clearly dictates otherwise. Thus, for example, reference to “a mouse” includes a plurality of such mice and reference to “the cytokine” includes reference to one or more cytokines and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

All publications mentioned herein are incorporated herein by reference for all relevant purposes, e.g., the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Example 1

In this study it is shown that RhoA and ζ PKC play diversified, yet necessary, roles in rapid LFA-1 triggering by chemokines in lymphocytes. RhoA controls both LFA-1 high affinity state and lateral mobility induction by chemokines through the engagement of distinct RhoA downstream effector domains. Blockade of RhoA downstream effector domain prevents the arrest of circulating naïve lymphocytes on ICAM-1-expressing high endothelial venules (HEV) in secondary lymphoid organs. These findings show that RhoA is critical in signal transduction pathways generated by chemokines and leading to LFA-1 activation.

Results

A novel method to analyze RhoA signaling activity. Three distinct domains of RhoA have been shown to activate individual downstream effectors. To block RhoA-dependent signaling in a domain-selective manner, fusion peptides were generated, including an N-terminal plasma membrane translocating sequence from the third helix of the homeodomain of Drosophila Melanogaster transcription factor Antennapedia, also called Penetratin-1 (P1), fused to the three distinct downstream effector domains of human RhoA, encompassing amino acids 23-40 (putative switch I region), 75-92 and 92-119.

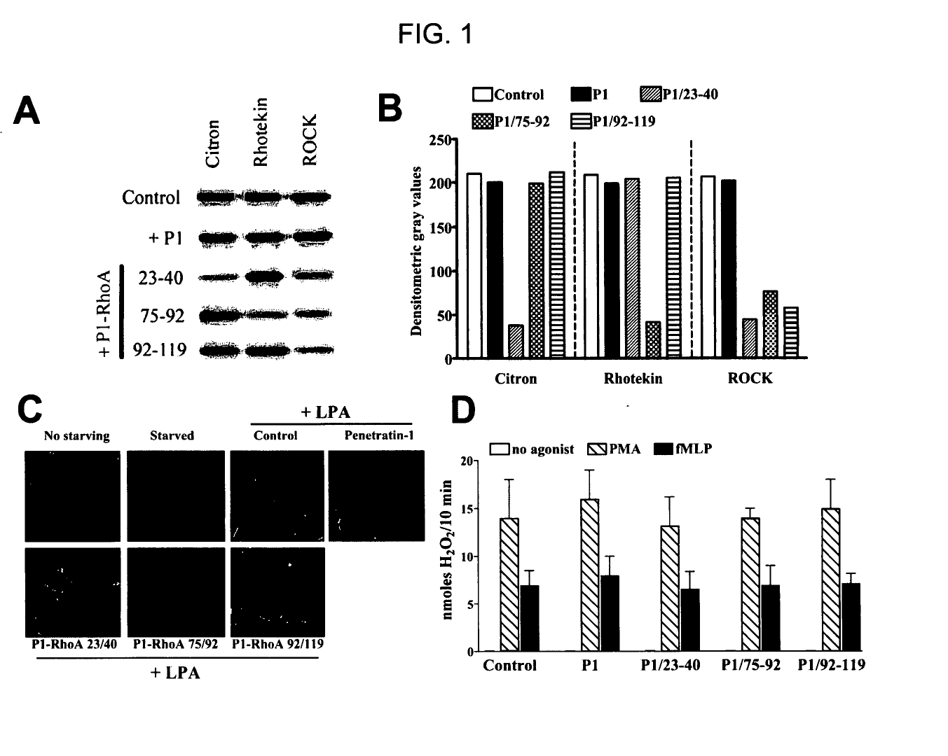

The ability of the domains to block RhoA interaction with specific effectors was evaluated. In pull-down assays, binding of RhoA to Citron was blocked by 23-40, but not 75-92 and 92-119, domain; binding to Rhotekin was blocked by 75-92, but not 23-40 and 92-119, domain; finally, binding to ROCK was inhibited by all the domains (FIG. 1A-B). These data show that soluble RhoA-derived effector domains may block RhoA interaction with specific effectors. As the three domains were able to significantly prevent RhoA interaction with ROCK, we could validate the biological activity of the domains by testing the capability of P1-RhoA fusion domains to interfere with the accumulation of actin stress fibers induced by lysophosphatidic acid (LPA) in fibroblasts, a phenomenon dependent on RhoA-activated Rho-kinase (ROCK).

Pretreatment of fibroblasts with each domain almost completely prevented the accumulation of stress fibers upon LPA triggering. In contrast, the peptide encompassing only the P1 sequence was completely ineffective (FIG. 1C). Inhibition was dose-dependent with significant effects starting at 10 μM. These results are consistent with the known involvement of these RhoA sites in ROCK activation and with the result of our pull down experiment. In contrast, LPA-induced membrane ruffling, which is Rac1-dependent, was unaffected by pretreatment with the fusion domains. Furthermore, in human polymorphonuclear neutrophils, RhoA domains were unable to block the phorbol myristate acetate (PMA)- and formyl-Met-Leu-Phe (fMLP)-induced activation of the superoxide forming complex NADPH-oxidase, whose activation relies on rac activity (FIG. 1D). Together, these data show that P1-RhoA fusion effector domains are effective selective inhibitors of RhoA-induced signaling events.

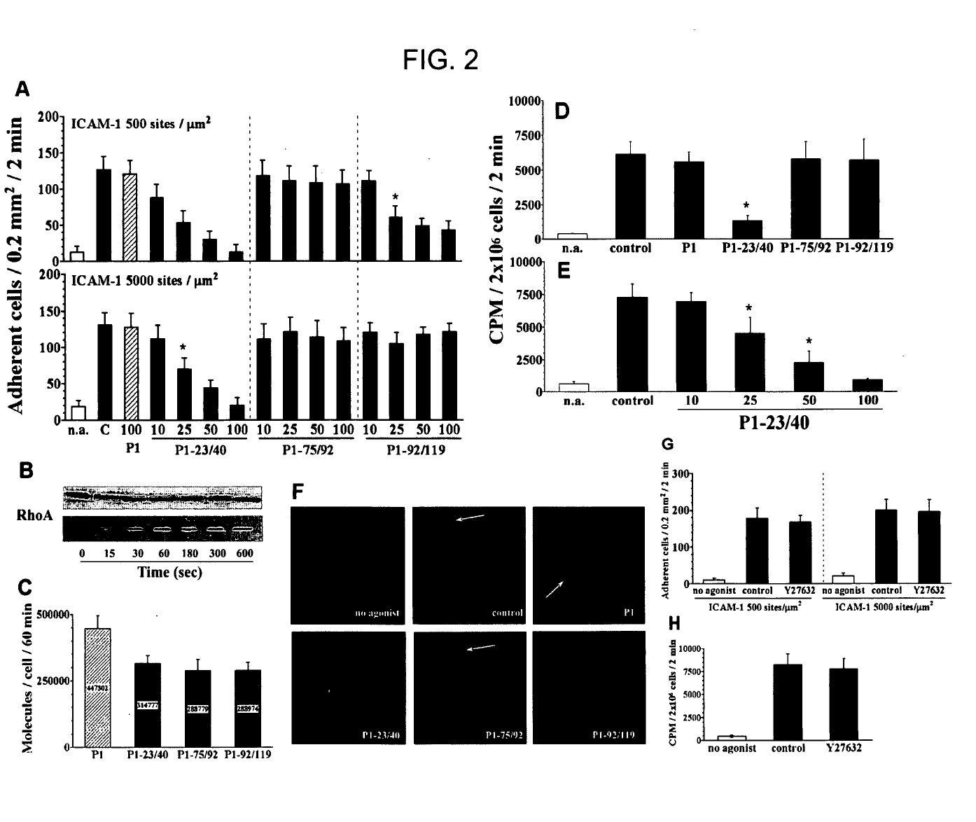

Distinct RhoA effector domains control chemokine-induced LFA-1-dependent rapid lymphocyte adhesion to ICAM-1. To evaluate the role of RhoA in the complex modality of LFA-1 activation by chemokines, we first investigated the involvement of RhoA in CCL21-induced rapid LFA-1-dependent lymphocyte adhesion to ICAM-1 immobilized at either low or high site densities. When lymphocytes were stimulated to adhere to low density of ICAM-1 (−500 sites/μm²), both 23-40 and 92-119 P1-RhoA fusion domains prevented in a dose-dependent manner rapid adhesion induced by CCL21 (FIG. 2A). In contrast, the 75-92 P1-RhoA fusion domain or the P1 peptide were unable to block rapid adhesion triggering. However, on high density of ICAM-1 (˜5000 sites/μm²), only the 23-40 P1-RhoA fusion domain blocked adhesion triggering in a dose-dependent manner, whereas P1, 75-92 and 92-119 P1-RhoA fusion domains were ineffective (FIG. 2A). None of the used fusion domains interfered with intracellular calcium increase induced by CCL21 thus ruling out potential nonspecific effects. RhoA involvement in CCL21-induced LFA-1-dependent lymphocyte adhesion was further corroborated by biochemical analysis showing that CCL21 activated RhoA within seconds, thus displaying kinetics consistent with rapid LFA-1 triggering (FIG. 2B). To exclude that the difference between P1-RhoA fusion domains in lymphocytes could be due to unequal accumulation of the fusion domains into the cell, we calculated the number of molecules of different fusion domains loaded per single cell. Although the P1 peptide loaded more efficiently, all three different P1-RhoA fusion domains showed a similar capability to accumulate inside lymphocytes (FIG. 2C). Together, these data show that RhoA controls LFA-1 activation by CCL21 through the signaling activity of two distinct effector domains.

Distinct RhoA effector domains control the different modalities of LFA-1 activation by chemokines. Chemokine-triggered lymphocyte arrest in condition of variable density of ICAM-1 relies on distinct intracellular signaling pathways specifically controlling the two modalities of LFA-1 activation. Thus, the previous data strongly suggest that RhoA activates both modalities of LFA-1-activation through the distinct signaling activity of 23-40 and 92-119 effector domains. To test this hypothesis, we evaluated the capability of different P1-RhoA fusion domains to interfere with LFA-1 high affinity state and rapid lateral mobility triggering by chemokines. Pretreatment with the P1 control peptide or the 75-92 and 92-119 P1-RhoA fusion domains did not prevent LFA-1 high affinity state triggering (FIG. 2D). In contrast, pretreatment of lymphocytes with the 23-40 P1-RhoA fusion domain prevented heterodimer high affinity state induction by CCL21 (FIG. 2D). Inhibition of LFA-1 high affinity state triggering by the 2340 P1-RhoA fusion domain was dose-dependent and almost complete, with a maximum blockade of about 90% (FIG. 2E).

We next determined the involvement of RhoA in the induction of rapid LFA-1 lateral mobility by CCL21. Pretreatment with the P1 control peptide or with the 75-92 P1-RhoA fusion domain did not prevent LFA-1 rapid lateral mobility induced by CCL21. In contrast, pretreatment with both 23-40 and 92-119 P1-RhoA fusion domains prevented rapid generation of LFA-1 clusters (FIG. 2F and Table 1).

Taken together, these data show that RhoA controls both modalities of chemokine-induced rapid LFA-1 activation by means of distinct effector regions. Interestingly, the 75-92 effector domain, involved in ROCK activation, does not appear to have a role in chemokine-induced integrin activation. Notably, 23-40 and 92-119 RhoA domains have been also implicated in ROCK activation. Therefore, we wished to determine whether ROCK could be an effector in RhoA-dependent LFA-1 rapid activation by chemokines. Pretreatment of lymphocytes with Y27632, a specific ROCK inhibitor, did not inhibit CCL21-induced lymphocyte adhesion to ICAM-1 immobilized either at low or high site density (FIG. 2G). Furthermore, neither LFA-1 affinity triggering (FIG. 2H) nor induction of lateral mobility were blocked by Y27632. These data rule out ROCK as a possible downstream signaling effector linking RhoA to rapid LFA-1 activation by chemokines.

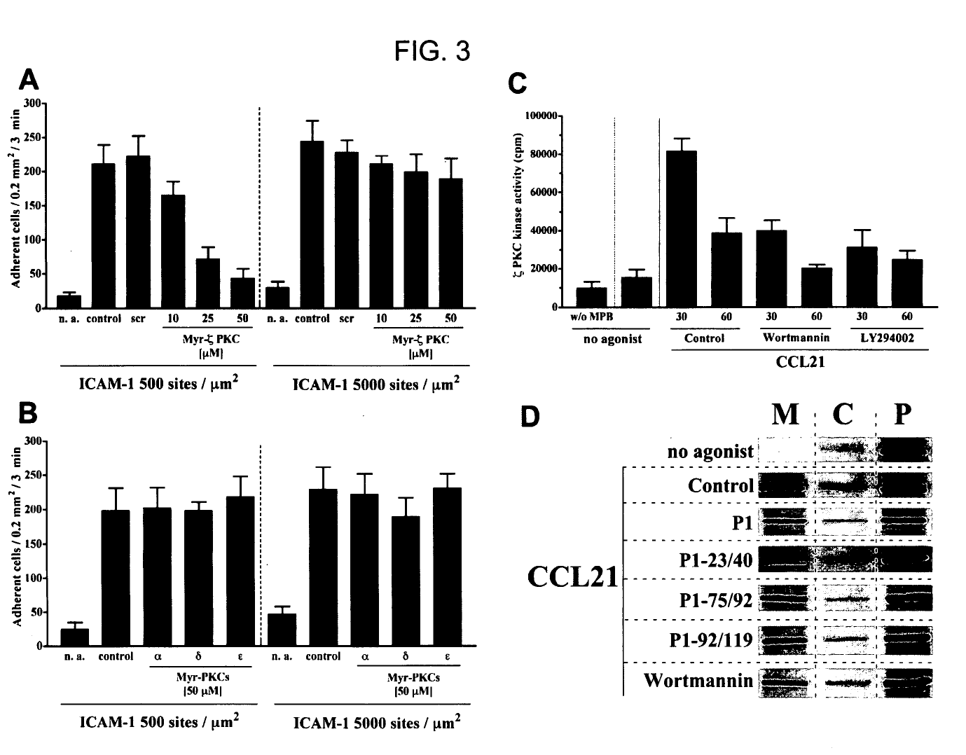

The atypical ζ PKC is involved in chemokine-induced LFA-1-dependent rapid lymphocyte adhesion to ICAM-1. Previous data proposed ζ PKC as potential RhoA effector implicated in Mac1 activation by chemoattractants in polymorphonucelar cells (PMNs). To evaluate the role of ζ PKC in rapid LFA-1 triggering by chemokines in lymphocytes, we used myristoylated peptides with sequence identical to the pseudosubstrate inhibitory region of ζ PKC. As shown in FIG. 3A, blockade of ζ PKC activity inhibited in a dose-dependent manner adhesion triggering to ICAM-1 induced by CCL21. Notably, the inhibitory effect of the ζ PKC peptide was evident only on low density of ICAM-1. Moreover, a control peptide, with a scrambled sequence (FIG. 3A), as well as peptides with sequence identical to the pseudosubstrate region of α, δ and ε PKCs (FIG. 3B) were completely unable to prevent adhesion triggering either on low or high density of ICAM-1.

To further corroborate the involvement of ζ PKC in signaling pathways generated by chemokines and leading to LFA-1 activation, we analyzed the activation state of ζ PKC upon CCL21 stimulation. As shown in FIG. 3C, CCL21 induced a consistent and rapid increase of ζ PKC kinase activity. We also evaluated the intracellular distribution of ζ PKC. As shown in FIG. 3D, in resting lymphocytes ζ PKC was mainly associated with the particulate fraction, whereas it was almost absent in the cytosol and plasma membrane. Upon triggering with CCL21, ζ PKC rapidly translocated to the plasma membrane fraction. These data show that the atypical ζ PKC is the only PKC isoform expressed in lymphocytes selectively involved in rapid LFA-1 triggering by chemokines. Importantly, ζ PKC seems relevant only to adhesion to low density of ICAM-1.

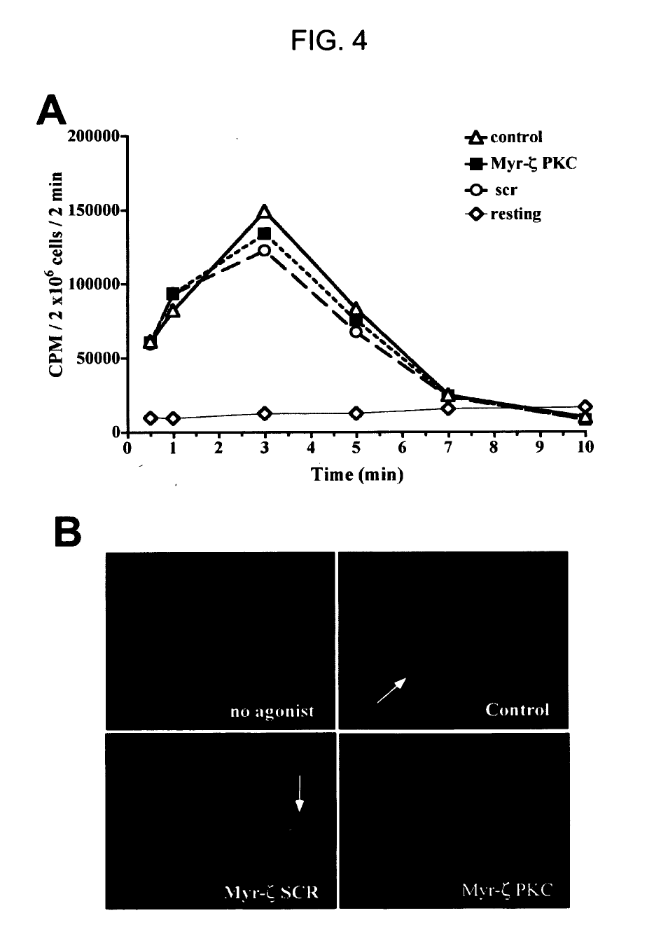

The atypical ζ PKC controls LFA-1 lateral mobility but not high affinity state induction by chemokines. The previous data suggest a role for ζ PKC in rapid LFA-1 lateral mobility but not high affinity state induction by CCL21. To validate this hypothesis we determined the role of ζ PKC in LFA-1 high affinity state and lateral mobility induction by CCL21. As shown in FIG. 4A, pretreatment of lymphocytes with ζ PKC inhibitory peptides or with a scrambled peptide, did not prevent the rapid and transient induction of LFA-1 high affinity state induced by CCL21. α, δ and ε PKC inhibitory peptides were also unable to block LFA-1 high affinity state induction. However, confocal microscopy analysis showed that pretreatment of lymphocytes with the ζ PKC inhibitory peptide, but not with a scrambled peptide, prevents the accumulation of large LFA-1 clusters rapidly induced by CCL21 (FIG. 4B). α, δ and ε PKC blocking peptides were completely unable to inhibit LFA-1 cluster formation induced by CCL21.

Together, these data show that classical, novel and atypical PKCs isoforms expressed in lymphocytes are not involved in LFA-1 high affinity state triggering by CCL21. In contrast, the atypical isoform ζ PKC is necessary to rapid LFA-1 lateral mobility on the plasma membrane induced by CCL21.

The role of PI(3)K and RhoA in ζ PKC activation by chemokines. PI(3)K has been previously implicated in rapid LFA-1 lateral mobility as well as in ζ PKC activation. As shown in FIG. 3C, pretreatment of lymphocytes with Wortmannin or with LY234002, two PI(3)K specific inhibitors, partially prevented the increase of ζ PKC kinase activity induced by CCL21 (about 51% for Wortmannin and 62% for LY294002). In contrast, pretreatment with PI(3)K inhibitor did not affect ζ PKC translocation to the plasma membrane (FIG. 3D). Thus, PI(3)K partially mediates CCL21-induced increase of kinase activity but not translocation of ζ PKC to the plasma membrane in lymphocytes.

Chemoattractant-induced ζ PKC translocation to the plasma membrane relies on RhoA activity in PMNs (Laudanna et al., 1998). Having identified RhoA downstream effector domains critical to rapid LFA-1 triggering, we had the possibility to investigate the involvement of distinct RhoA effector domains in ζ PKC translocation induced by chemokines in lymphocytes. As shown in FIG. 3D, pretreatment of lymphocytes with P1 peptide or with 75-92 or 92-119 P1-RhoA fusion domains did not prevent ζ PKC translocation to the plasma membrane. However, pretreatment with the 2340 P1-RhoA fusion domain blocked ζ PKC translocation to the plasma membrane; the densitometric analysis showed a blockade of about 82%.

These data show that induction of ζ PKC kinase activity by CCL21 in lymphocytes partially depends on PI(3)K, whereas ζ PKC tranlocation to the plasmamembrane is almost completely controlled by a restricted subset of RhoA-dependent signaling activated by the 23-40 downstream effector region.

RhoA-dependent LFA-1 high affinity state is the modality of integrin activation controlling lymphocyte homing in vivo. The data presented above establish diversified roles for RhoA and ζ PKC in controlling distinct modalities of LFA-1 activation. Previous data suggested a role for LFA-1 triggered to high affinity state in lymphocyte homing to secondary lymphoid organs. However, a formal demonstration has never been provided. The definition of the role of 23-40 RhoA domain in LFA-1 high affinity state triggering by chemokines prompted us to pursue a formal demonstration of the role of LFA-1 triggering to high affinity state in the recruitment of circulating lymphocytes in vivo.

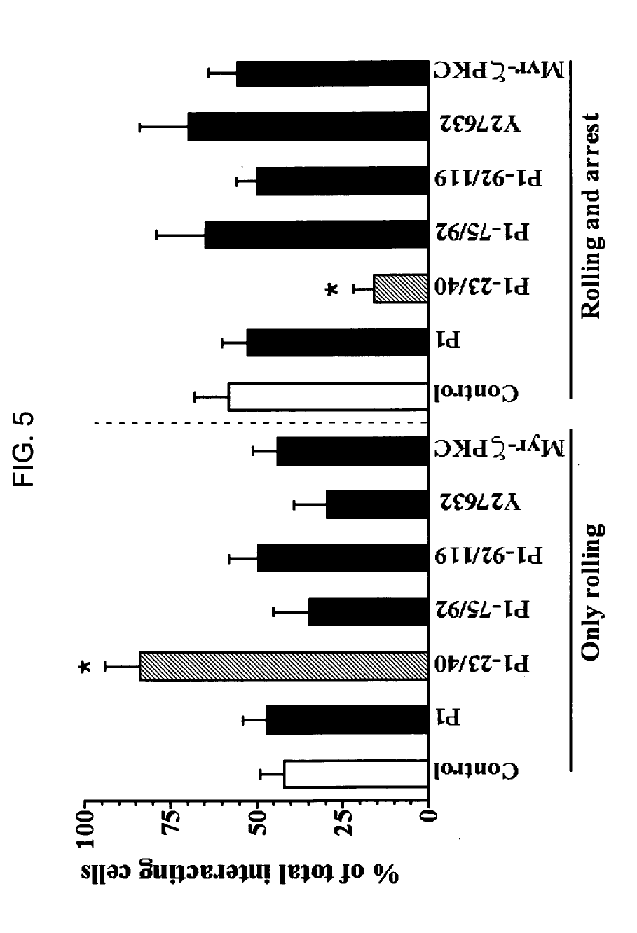

Pretreatment of lymphocytes with the P1 control peptide or with the 75-92 or 92-119 P1-RhoA fusion domains did not affect rolling and arrest of circulating lymphocytes on high endothelial venules in the secondary lymphoid organ Peyer's patch (PP-HEV) (FIG. 5). Pretreatment with the 2340 P1-RhoA fusion domain did not influence lymphocyte tethering and allowed normal interaction with vessels. However, this fusion domain consistently inhibited the stable arrest of lymphocytes on PP-HEV, with about 75% of inhibition (P<0.01); the percentage of cells displaying only rolling increased, as expected. Notably, the site density of ICAM-1 presented to the interacting lymphocytes on PP-HEV was previously shown to be extremely high (about 14,000 site/m²), a condition in which LFA-1 accelerated lateral mobility is not required to rapid arrest. Indeed, the 92-119 P1-RhoA fusion domain, which only affected LFA-1 rapid lateral mobility induced by CCL21, had no effect on lymphocyte arrest in PP-HEV. As the 23-40 RhoA effector domain controls the induction of LFA-1 conformational change by CCL21 (see FIG. 2), these data demonstrate that RhoA-controlled heterodimer high affinity state is the modality of LFA-1 activation critically required to rapid arrest of circulating lymphocytes in PP-HEV.

The role of ROCK kinase and ζ PKC in rapid lymphocytes recruitment to HEV was tested. Lymphocytes pretreated with Y27632 rolled and adhered normally in HEV. Moreover, pretreatment of lymphocytes with the ζ PKC inhibitory peptide did not affect the capability of lymphocytes to roll and arrest on HEV (FIG. 5). These data are consistent with the inability of these inhibitors to prevent LFA-1 affinity triggering by CCL21 and exclude a participation of ROCK and ζ PKC in signaling events leading to lymphocyte rapid arrest in PP-HEV.

Chemokines are powerful physiological activators of lymphocyte integrins. Chemokines play a dual role in LFA-1-mediated rapid lymphocyte adhesion by inducing LFA-1 high affinity state and accelerated lateral mobility. The intracellular signaling events differently controlling this complex phenomenon are identified herein, using the CCR7 ligand CCL21, which directs T-lymphocyte arrest in secondary lymphoid organs (identical results were obtained with CCL19 and with the CXCR4 ligand CXCL12).

It is shown that: (1) RhoA controls LFA-1 high affinity state triggering by chemokines; (2) RhoA also controls LFA-1 rapid lateral mobility induced by chemokines; (3) the signaling activity of two distinct RhoA effector regions controls LFA-1 activation by chemokines; (4) the atypical ζ PKC is critical to LFA-1 lateral mobility but not to high affinity state triggering; (5) chemokine-induced ζ PKC kinase activity and translocation to the plasma membrane depend respectively on PI(3)K and on the signaling activity of the 23-40 RhoA effector region; (6) rapid arrest of circulating lymphocytes on HEV in secondary lymphoid organs critically depends on the induction of LFA-1 high affinity state.

RhoA and the modality of LFA-1 activation by chemokines. It is shown that plasma membrane translocating RhoA-derived effector domains are useful tools to study RhoA-dependent signaling events in a domain-selective manner. The inhibitory activity of the domains is likely due to interference with the plasma membrane docking function of small GTPases, a step required to full activation of downstream effectors. By using these tools, we analyzed the role of RhoA in LFA-1-dependent rapid lymphocyte adhesion.

The data show that RhoA controls LFA-1 conformational change and lateral mobility by chemokines through the distinct, yet complementary, activity of two effector domains, encompassing amino acids 2340 and 92-119. The 92-119 domain is exclusively involved in LFA-1 lateral mobility induction. In contrast, the 23-40 RhoA domain participates in heterodimer lateral mobility regulation and is also critical to the induction of LFA-1 high affinity state.

This latter finding is of particular importance. The blocking activity of the P1-2340 fusion domain on LFA-1 affinity triggering by CCL21 highlighted the critical regulatory role of RhoA in LFA-1 high affinity triggering. RhoA is the first example of signaling molecule controlling the rapid induction of integrin high affinity state under physiological conditions. Importantly, blockade of 23-40 RhoA effector domain prevented LFA-1-dependent arrest of naïve lymphocytes on PP-HEV. Although 23-40 RhoA domain also controls LFA-1 lateral mobility, blockade of the 92-119 domain, which is only involved in heterodimer lateral mobility, did not interfere with arrest of naïve lymphocytes on PP-HEV. This shows that lymphocytes rely on activation of LFA-1 high affinity state to home to secondary lymphoid organs. This provides the first demonstration of the physiological role of the inside-out signal-dependent induction of LFA-1 high affinity state in vivo.

Biochemical analysis showed a rapid and prolonged RhoA activation by CCL21, with RhoA remaining in an active state for at least 10 minutes. Notably, the induction of LFA-1 high affinity state by chemokines displays transient kinetics, with integrin affinity completely down-modulated within few minutes. This suggests that down-modulation of LFA-1 high affinity state, which temporally correlates with down-modulation of lymphocyte rapid adhesion to ICAM-1, does not simply rely on RhoA inactivation. Thus, it is possible that chemokines generate signaling pathways able to actively counterbalance RhoA-dependent pathways leading to LFA-1 conformational changes. Notably, the activation of H-ras and the dependent MAP-kinase prevents the induction of LFA-1 high affinity state by the chemokine CXCL12. Thus, a temporally and spatially regulated balance between the signaling activities of H-ras and RhoA could regulate the dynamics of LFA-1 high affinity state activation and dependent rapid adhesion.

ζ PKC and the modality of LFA-1 activation by chemokines. The involvement of the atypical ζ PKC in LFA-1 activation by chemokines was determined. ζ PKC had no role in LFA-1 high affinity state triggering by CCL21. However, ζ PKC was found to be critical to rapid LFA-1 lateral mobility. Of interest, we also found that the slow LFA-1 clustering induced by PMA is sensitive to ζ PKC blockade. Accordingly, we previously showed that PMA, although not an allosteric activator of ζ PKC (atypical PKCs have incomplete C1 domain), may trigger ζ PKC translocation to the plasma membrane through RhoA activation. Notably, ζ PKC has a constitutive kinase activity and this implies that ζ PKC translocation to the plasma membrane may be sufficient to generate ζ PKC-dependent signaling events even in the absence of augmented kinase activity.

The ability of the 23-40 P1-RhoA fusion domain to block ζ PKC translocation suggests that ζ PKC may mediate, at least partially, the RhoA capability to control LFA-1 clustering. Moreover, this data suggests a direct interaction between RhoA and ζ PKC, thus establishing ζ PKC as a novel direct downstream effector of RhoA.

The complexity of the chemokine-induced pro-adhesive signaling network. The data presented here, together with previous reports, highlight the complexity of the signaling network generated by chemokines and leading to integrin activation. Chemokine-triggered LFA-1 lateral mobility is controlled at least by PI(3)K, Cytohesin-1, Rap1, ζ PKC and RhoA. Recruitment of Cytohesin-1 to the plasma membrane requires PI(3)K activity. Moreover, PI(3)K-derived PIP3 increases ζ PKC activity and the PIP3-dependent kinase PDK-1 is a direct activator of ζ PKC. Thus, PI(3)K appears to control both Cytohesin-1 and ζ PKC signaling activity leading to LFA-1 lateral mobility and clustering. However, regulation of ζ PKC does not seem to be dependent only on PI(3)K. Indeed, inhibition of PI(3)K does not totally prevent the increase of ζ PKC kinase activity induced by CCL21, and does not block translocation of ζ PKC to the plasma membrane. In contrast, ζ PKC translocation to the plasma membrane is critically dependent on signaling activity of RhoA 23-40 effector region. Altogether, these data show that ζ PKC translocation to the plasma membrane and increase of kinase activity are independently controlled by distinct signaling pathways.

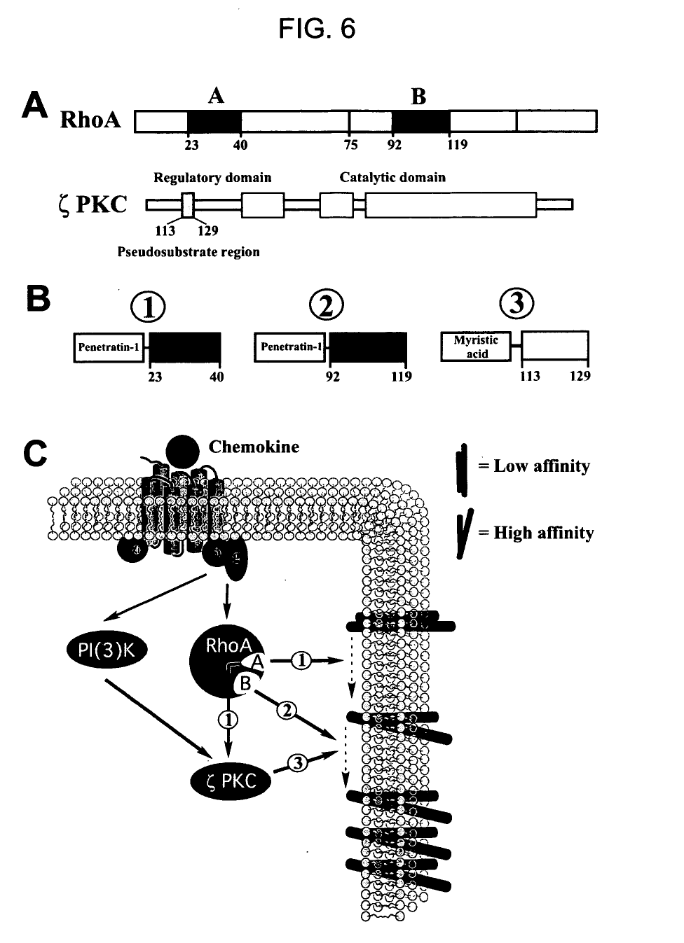

In conclusion, we show that RhoA and ζ PKC are critical components of the signaling network controlling the complex dynamic of activation of the β2 integrin LFA-1. The small GTP binding protein RhoA is a central point of diversification of signaling pathways controlling both the modalities of LFA-1 activation induced by chemokines. In contrast, ζ PKC is a point of convergence of signaling events controlling LFA-1 lateral mobility (FIG. 6). Importantly, we formally demonstrate the critical role of LFA-1 affinity triggering in lymphocyte in vivo homing. Selective blockade of the 92-119 RhoA domain could exclusively inhibit LFA-1 lateral mobility, without affecting lymphocyte homing to secondary lymphoid organs. Thus, the identification of discrete RhoA effector regions controlling distinct modalities of LFA-1 activation allows a more effective pharmacological approach to control leukocyte recruitment during inflammation.

Materials

PKC myristoylated pseudosubstrate peptides (synthesized at Stanford University PAN-facility) were dissolved before use at 1 mM concentration in PBS, pH 7.2. PMA, ketamine, xylosine were from Sigma; FCS was from Irvine; murine CCL21 and CXCL12 were form Peprotech (London, England); CMFDA, CMTMR and Alexa 488 labeling kit were from Molecular Probes; Texas Red-conjugated goat anti-rat antibody was from Jackson ImmunoResearch; murine ICAM-1 was purified from spleens.

Generation of Penetratin-1-RhoA fusion domains. The Penetratin-1 (P1) fusion protein expression vector pTm3Hb was kindly donated (CNRS, France). Oligonucleodites encompassing human RhoA bases 67-120 (aa 23-40), 223-276 (aa 75-92) and 274-357 (aa 92-119) were inserted between the BamHI and KpnI cloning sites. Recombinant proteins expressed in E. coli BL21(DE3)pLysS Gold were purified on heparin columns, dialyzed against PBS and stored at −80° C. Alternatively, P1, P1-23-40 and P1-75-92 fusion domains were synthesized by Sigma-Genosys. A glycine was inserted between P1 and RhoA domains to allow flexibility of the domains. Peptides displayed the following properties: P1: aa 16, mw 2246.78; P1-RhoA 23-40: aa 34, mw 4430.29; P1-RhoA 75-92: aa. 34, mw 4254.06; P1-RhoA 92-119: aa. 44, mw 5529.83. Lyophilized peptides were dissolved before the experiments. Preliminary experiments carried out with fluorescent peptides showed that P1 and P1-RhoA fusion domains accumulated in comparable manner in about 95% of lymphocytes.

Specificity of RhoA fusion domains and measurement of RhoA activation. The specificity of P1-RhoA fusion domains was evaluated by affinity-precipitation assay using the rho-binding domain (RBD) from Citron, Rhotekin and ROCK. Recombinant Val14-RhoA was loaded with 1 mM GTP at 37° C. for 90 min. 10 μg of Citron, Rhotekin or ROCK GST-RBD conjugated with glutathione beads were mixed with 5 μg of GTP-Val14-RhoA in 50 □g of 50 mM Tris-HCl (pH 7.4), 5 mM MgCl₂, 0.5 mM GTP, 2 mg/ml BSA (binding buffer). Binding was for 60 min. at 15° C. The beads were washed twice with binding buffer and subjected to SDS-polyacrylamide gel electrophoresis on a 12% gel. RhoA background binding to glutathione beads was negligible. In case of experiments in presence of P1-RhoA fusion domains, the domains were previously dissolved in binding buffer at 1 mg/ml.

RhoA activation by CCL21 was evaluated by using the rho-binding domain (RBD) from Rhotekin. Lymphocytes were lysed on ice in 0.5 ml of 100 mM HEPES buffer, pH 7.5, 1% Triton X-100, 1% de-oxycholate, 0.1% SDS, 500 mM NaCl, 10 mM MgCl₂, 2 mM EGTA, 2 mg/ml BSA, 20 mM benzamidine, containing the Complete™ protease inhibitor cocktail from Roche. Equal amount of lysates were incubated with GST-RBD (20 μg) beads for 45 min. at 4° C. Bound GTP-RhoA was identified by Western blotting using a monoclonal antibody from Santa Cruz.

Evaluation of actin stress fibers content. Swiss 3T3 mouse fibroblasts were maintained in DMEM containing 10% FCS for 6 days and then starved for 12 hours. Actin stress fibers content was evaluated in permeabilized cells with TRITC-labeled phalloidin. Analysis was performed by using a Zeiss LSM confocal microscope.

NADPH-oxidase activation. Activation of neutrophil NADPH-oxidase was evaluated by measuring reduction of dihydrorhodamine induced by superoxide anion-derived hydrogen peroxide, as previously reported. Human neutrophils were stimulated under stirring at 37° C. Time-course of hydrogen peroxide-induced dihydroRhodamine reduction was evaluated at the spectrofluorimeter with 505 of excitation wavelength and 534 of emission wavelength.

In vitro rapid adhesion assay on ICAM-1. ICAM-1 was purified from mouse spleens and adhesion assays were performed as previously reported. Briefly, primary naïve lymphocytes (about 70% T-, 30% B-cells) were isolated from peripheral and mesenteric lymph nodes and Peyer's patches from young BALB/c mice (Harlan, Italy). Adhesion assays were performed on eighteen well glass slides coated overnight at 4° C. with purified mouse ICAM-1; site density per square micrometer of immobilized ICAM-1 was calculated as reported.

Calculation of internalization efficiency of the Penetratin-1-RhoA fusion domains. P1 and P1-RhoA fusion domains were labeled with ¹²⁵I using the Bolton-Hunter reagent (Pierce) following instructions from the manufacturer. Loading efficiency at room temperature or 37° C. was identical and was linear between 10 and 150 μM. At 4° C. loading efficiency was reduced of about 40%. Presence of 10% serum did not affect protein loading. After loading, cells were rapidly washed three times in PBS, mildly treated with trypsin to remove peptides eventually adsorbed to the outer plasma membrane and the internalized radioactivity was measured with a gamma counter. Treatment with trypsin removed no more than 5-10% of total radioactivity. Specific activity was converted in CPM/molecules and then the number of molecules per cell was calculated.

Measurement of LFA-1 high affinity state. Induction of LFA-1 high affinity state by CCL21 was evaluated by measuring binding of soluble ¹²⁵I-ICAM-1. Briefly, ICAM-1 was iodinated with ¹²⁵NaI by the Chizzonite method. The binding assay was performed at 37° C. in a 500 μl Eppendorf tube. 40 μl of lymphocyte suspension (5×10⁷/ml in PBS containing 1 mg/ml BSA, 2 mM MgCl₂, 1 mM CaCl₂, 1 mM D-glucose, pH 7.2) were directly layered on 100 μp oil cushion of 2/1 dibutyl/dioctyl phthalates. Lymphocytes were stimulated with 10 μl of PBS containing chemokine (6 μM) and ¹²⁵I-ICAM-1 (5×10⁵ CPM corresponding approximately to 2 μg of ¹²⁵I-ICAM-1). The binding reaction was stopped by rapid centrifugation in microfuge. Radioactivity bound to lymphocytes was counted with a gamma counter.

Evaluation of LFA-1 distribution on plasma membrane. Analysis and quantification of chemokine-induced LFA-1 surface distribution was determined by confocal microscopy, following the same procedure previously described. Briefly, lymphocytes were stimulated in suspension under stirring and then immediately fixed in 1% ice-cold paraformaldehyde in PBS, pH 7.4, for 10 min. Cell were washed and incubated with 10 μg/ml of TIB213 rat anti-mouse LFA-1 (ATCC) for 30 min on ice, washed 3 times and then incubated 30 min with Texas Red-conjugated goat anti-rat secondary antibody. The washed cells adhered for 30 min at 4° C. on 0.1% poly-L-lysine coated round 13 mm glass coverslips and were analyzed with a Carl Zeiss LSM 510 confocal imaging system, with a 63×C-Apochromat objective (NA 1.2). Definition and quantitative analysis of “disperse” and “clustered” morphologies of LFA-1 distribution was as previously described.

Measurement of ζ PKC kinase activity. The assay was performed as previously described. Briefly, lymphocytes were stimulated under stirring with agonists at 37° C. Stimulation was stopped with lysis buffer containing 50 mM Tris-HCl, pH 7.5, 1% Triton X-100, 0.01% SDS, 150 mM NaCl, 50 mM NaF, 10 mM sodium pyrophosphate, 1 μM phenylarsine oxide, containing the Complete™ protease inhibitor cocktail from Roche. After 30 min. on ice, lysates were centrifuged at 16,000 g for 1 min. to remove cell debris. Rabbit polyclonal anti ζ PKC (1 μg) or control rabbit serum was added to equal amount of cell lysates, followed by immunoprecipitation with trisacryl protein A. Equal amounts of ζ PKC were immunoprecipitated as confirmed by Western blot analysis. After four washing, immunoprecipitates were subjected to the kinase reaction for 30 min. at 30° C. in 50 μl of kinase buffer containing 0.5 mM EGTA, 10 mM MgC₂, 20 mM HEPES, pH 7.4, 50 μM ATP, 5 μCi [γ-³²P]ATP and 2 μg myelin basic protein (MBP) as a substrate. The reaction was stopped by addition of 5% TCA and the reaction mixture was filtered through phosphocellulose paper. After four rinses with 1% phosphoric acid radioactivity on the filter was determined at a scintillation counter.

ζ PKC intracellular distribution. The assay was performed as previously described. Briefly, lymphocytes were stimulated under stirring with agonists at 37° C. Stimulation was stopped by diluting the cells in a 10 times larger volume of ice-cold PBS. Cells, resuspended in 1 ml of ice-cold PBS containing 8% sucrose, containing the Complete™ protease inhibitor cocktail from Roche, were sonicated and the homogenates were centrifuged at 800×g/10 min to remove nuclei and unbroken cells. The postnuclear supernatant was loaded on discontinuous sucrose gradient (50% sucrose, 30% sucrose) and centrifuged for 120 min. at 100,000×g. The light membrane fraction (plasma membrane) was collected in the 30% layer. Following SDS-PAGE on 10% acrylamide, proteins were electroblotted on nitrocellulose filters, probed with rabbit polyclonal antibodies anti ζ PKC (Santa Cruz Biotechnology), followed by goat polyclonal anti-rabbit HRP conjugated (Sigma) and developed using ECL (Amersham).

Intravital video microscopy analysis of lymphocyte-high endothelial venule interaction in Peyer's patches. Lymphocytes (5×10⁶/ml in DMEM without sodium bicarbonate supplemented with 20 mM Hepes, 5% FCS, pH7.1) were labeled with either CMFDA or CMTMR for 30 min at 37° C. 30×10⁶ labeled cells were injected iv. In situ videomicroscopic analyses were carried out in high endothelial venules (HEV) in the secondary lymphoid organ Peyer's patch (PP) as previously described. Cell behavior was analyzed over a period of 20-30 min starting at 2 minutes after iv injection. Interactions of ≧1 s were considered significant and were scored. Cells were considered to be interacting whether they rolled, arrested or both. Lymphocytes that remained firmly adherent on venular wall for ≧10 s were considered arrested.

### Example 2

### Effect of Peptides on Lymphocytre Recruitment in the Brain

The RhoA derived peptides described in Example 1 were tested for their ability to inhibit Ag-stimulated lymphocyte (autoreactive, encephalitogenic T lymphocytes) recruitment in inflamed brain by looking in vivo cell behavior with intravital microscopy, as described in Example 1. This in vivo model simulates, in mouse, human Multiple Sclerosis.

It was found that the 2340 peptides blocks recruitment, as expected due to the role of LFA-1 affinity triggering in arrest. However, and importantly, it was also found that the 92-119 peptide greatly prevents the arrest of Ag-stimulated lymphocyte in inflamed microvessels. This domain is not involved in naive lymphocyte arrest in secondary lymphoid organ HEVs). As 92-119 blocks LFA-1 lateral mobility, these data confirm the role of integrin lateral mobility in adapting cell arrest in vessels expressing limiting amount of integrin ligand (as during the different phases of inflammation).

Thus, integrin lateral mobility seems to have a dual role: rapid generation of clusters, to facilitate outside-in signaling, and adaptation of adhesion during the inflammation. The capability of the 92-119 and 23-40 to block EAE (animal model of MS) is also tested.

Of particular interest (both at theoretical as well as pharmacological level) is the inhibitory effect of the 92-119 peptide, which does not block in vivo physiologic homing to secondary lymphoid organs, but does prevent recruitment of autoreactive lymphocytes to brain. This clearly suggests the usage of this peptide as pharmacological treatment for multiple sclerosis.

1. A peptide comprising a domain of RhoA conjugated to a transport domain, having the formula TXR, where T is a transport domain, X is a linker and R is a domain of RhoA.

2. The peptide according to claim 1, wherein said RhoA is a human RhoA protein.

3. The peptide according to claim 2, wherein said RhoA domain is selected from Domain I, II or III.

4. The peptide according to claim 3, wherein said RhoA domain comprises SEQ ID NO:2.

5. The peptide according to claim 3, wherein said RhoA domain comprises SEQ ID NO:3.

6. The peptide according to claim 3, wherein said RhoA domain comprises SEQ ID NO:4.

7. The peptide according to claim 1, wherein said transport domain is a peptide domain.

8. The peptide according to claim 7, wherein said transport domain is fused to said RhoA domain through said linker.

9. The peptide according to claim 8, wherein said linker is a peptide linker.

10. The peptide according to claim 1, further comprising a pharmaceutically acceptable excipient.

11. A method of inhibiting RhoA signaling in a mammalian cell, the method comprising:
contacting said cell with a peptide according to claim 1.

12. The method according to claim 11, wherein said RhoA signaling controls LFA-1 high affinity state and/or lateral mobility induction.

13. The method according to claim 12, wherein said cell is a leukocyte.

14. The method according to claim 11, wherein said cell is cultured in vitro.
